(19) United States
(12) Patent Application Publication
(10) Pub. No.: US 2008/0033433 A1
(43) Pub. Date: Feb. 7, 2008

(54) DYNAMIC SPINAL STABILIZATION DEVICE

(76) Inventor: Dante Implicito, Franklin Lakes, NJ (US)

Correspondence Address: KENYON & KENYON LLP, 1500 K STREET N.W., SUITE 700, WASHINGTON, DC, 20005, US

(21) Appl. No.: 11/831,494

(22) Filed: Jul. 31, 2007

Related U.S. Application Data
(60) Provisional application No. 60/834,495, filed on Aug. 1, 2006.

(51) Int. Cl. A61B 17/58; A61B 17/56

(52) U.S. Cl. 606/61; 606/73

(57) ABSTRACT

The present invention regards linking spinal vertebrae to provide support and alignment of one or more vertebrae. The various systems, methods, devices and kits that embody the invention may employ a vertebrae crossover connector to link or otherwise connect one or more vertebrae in a column of vertebrae. These connectors may be positioned between anchors added to the vertebrae and may span the center of the spinal column when the connectors are positioned between the anchors. In certain embodiments each crossover connector may include one or more stretchable couplings that can stretch and resist tensile loads placed on them when the spinal column is stationary and in various ranges of motion. The crossover connector may also include a main body that may resist compressive loads placed on the crossover connector when the spinal column is in various ranges of motion in certain positions.

### FIELD OF THE INVENTION

The present invention relates to spinal vertebrae connections. More specifically, the present invention relates to methods, systems, apparatus, and kits that may function to stabilize vertebrae of the spine.

### BACKGROUND OF THE INVENTION

The human spinal column consists of a series of thirty-three stacked vertebrae. Each vertebrae is separated by a disc and includes a vertebral body having several posterior facing structures. These posterior structures include pedicles, lamina, articular processes, and spinous process. The articular processes, which function as pivoting points between vertebrae, include left and right superior and inferior processes. The superior and inferior processes of adjacent vertebrae mate with each other to form joints called facet joints. In a typical pair of vertebrae, the inferior articular facet of an upper vertebrae mates with the superior articular facet of the vertebra below to form a facet joint.

The facet joints of the spinal column contribute to the movement and the support of the spine. This movement and rotation is greatest in the cervical (upper) spine region and more restrictive near the lumbar (lower) spine region. In the cervical region of the spine, the articular facets are angled and permit considerable flexion, extension, lateral flexion and rotation. In the thoracic region, the articular facets are oriented in the coronal plane and permit some rotation, but no flexion or extension. In the lumbar region of the spinal column, the articular facets are oriented in a parasagittal plane and permit flexion, extension and lateral bending but they limit rotation.

Through disease or injury, the posterior elements of the spine, including the facet joints of one or more vertebrae, can become damaged such that the vertebrae no longer articulate or properly align with each other. This can result in a misaligned anatomy, immobility, and pain. As such, it is sometimes necessary to remove part or all of the facet joint with a partial or full facetectomy. Removal of facet joints, however, destabilizes the spinal column as adjacent stacked vertebrae can no longer fully interact with and support each other.

One way to stabilize the spinal column after removal of facet joints or other posterior elements of the spine is to vertically rigidly fix adjacent stacked vertebrae through bone grafting and/or rigid mechanical fixation assemblies. In each case, the adjacent vertebrae are rigidly fixed to one another through a medical procedure. In these fixed systems, the spine looses flexibility as two previously moveable vertebrae are fused a certain distance apart from one another and, consequently, function and move as a single unit.

### SUMMARY OF THE INVENTION

The present invention regards linking spinal vertebrae to stabilize vertebrae and preserve physiological spinal movement. Preferably, the crossover connector simulates the natural functions of intact facet joints, such as accommodating flexion and extension, and limiting rotation. The various systems, methods, devices, and kits that embody the invention may employ a vertebrae crossover connector to link or otherwise connect vertebrae in a spinal column. These connectors may be positioned during a procedure between anchors inserted in vertebrae such that they crossover the spinal canal. In certain embodiments, each crossover connector may include one or more stretchable couplings that can stretch and resist tensile loads placed on them by the anchors coupled to the spinal column when the anchors move apart from one another. The crossover connector may also include a main body that may bear compressive loads placed on the connector by these same anchors when they move towards one another. The main body of one or more crossover connectors may be linear, bowed and may even form an “X” shape in some instances. The X shape may be formed by the body itself as well as when multiple connectors are surgically placed across one another between vertebrae anchors. There are numerous other configurations of the crossover connector and the components that comprise it.

The present invention may be embodied in a spinal stabilization kit. This kit may include: (a) a crossover connector having a main body and one or more stretchable couplings; (b) a plurality of spacers that may be used to change the length and configuration of the main body; and (c) a plurality of screws that may be used to anchor the crossover connector to the vertebrae. These kits may also include instructions directed toward the proper handling and use of the components of the kit.

Methods that employ the invention may include procedures that position pedicle screws into two or more vertebrae of a spinal column and mechanically link these screws with one or more crossover connectors. These connectors may be positioned such that the connectors crossover a center line of the spinal column defined by the spinal canal. For example, in a pair of lower vertebrae, a pedicle screw in the lower vertebrae may be linked to a pedicle screw opposite it in the upper vertebrae by the crossover connector. Thus, the connector will connect the upper and lower vertebrae and will also cross over the spinal canal. In this example another crossover connector may also be installed between another diagonally opposed set of anchors positioned on the lower and upper vertebrae. If this is done, an X shape is formed by the crossover connectors. As noted, this X shape may also be formed by a single integrated crossover connector that spans four anchors and two vertebrae. A method of the invention may also include measuring the distance between anchors that have been installed in one or more vertebrae and adjusting the crossover connector to accommodate these measured distances, the configuration of the installed anchors or both. These adjustments may be carried out by adding spacers to one or more ends of the crossover connectors they may also be carried out by reconfiguring the crossover connector in some other fashion. More, fewer and other actions that embody the invention in addition those identified herein are also possible.

### BRIEF DESCRIPTION OF THE DRAWINGS

The present invention is described throughout, including the following detailed description and the accompanying drawings. The description and the drawings are examples of the invention, numerous other embodiments are also possible. The drawings that accompany this specification are as follows:

FIG. 1 is a posterior or rear view of a spinal column having multiple anchors and a crossover connector in accord with the present invention.

FIG. 2 is a plan view of a main body of a crossover connector in accord with the present invention.

FIG. 3 is a sectional plan view of the main body from FIG. 2 also including a stretchable coupling.

FIG. 4 is a plan view of two crossover connectors, forming an X shape, in accord with the present invention.

FIG. 5 is a sectional plan view of the main body from FIG. 2 also including a stretchable coupling having a frame linking chords of the stretchable coupling.

FIG. 6 is a perspective view of a main body of a crossover connector in accord with the present invention.

FIG. 7 is a sectional view of the main body of FIG. 6 along line 7-7.

FIG. 8 is a sectional view of the main body of FIG. 6 along line 8-8.

FIG. 9 is a perspective side view of a spacer that may be coupled to the main body of a crossover connector.

FIG. 10 is a plan view showing an example of a mating relationship that may be used between a spacer and anchor in accord with the present invention.

FIG. 11 is a perspective view of a pedicle screw as may be employed as an anchor in accord with the present invention.

FIGS. 12a-12d are a series of block illustrations showing the pushing and pulling that may occur on the crossover connector when the connector is anchored to two spinal vertebrae and the anchors move relative to one another.

FIG. 13 is a posterior view of a spinal column and crossover connector corresponding to FIG. 12b, where the spinal column is flexed such that the anchors move away from one another.

FIG. 14 is a posterior view of a spinal column and crossover connector corresponding to FIG. 12c, where the spinal column is flexed such that the anchors move towards one another.

FIG. 15 is a posterior view of a spinal column and crossover connector corresponding to FIG. 12d, where the spinal column is rotated such that the anchors change position relative to one another.

FIG. 16 is a method that may be carried out in accord with the present invention.

### DETAILED DESCRIPTION

In embodiments of the present invention, crossover connectors may be employed to stabilize the spinal column and to facilitate motion of the spinal column. The invention may be employed after the removal of posterior portions of vertebrae and/or after some trauma or deterioration has occurred to the vertebrae. The invention may be used at other times and in other clinical situations as well.

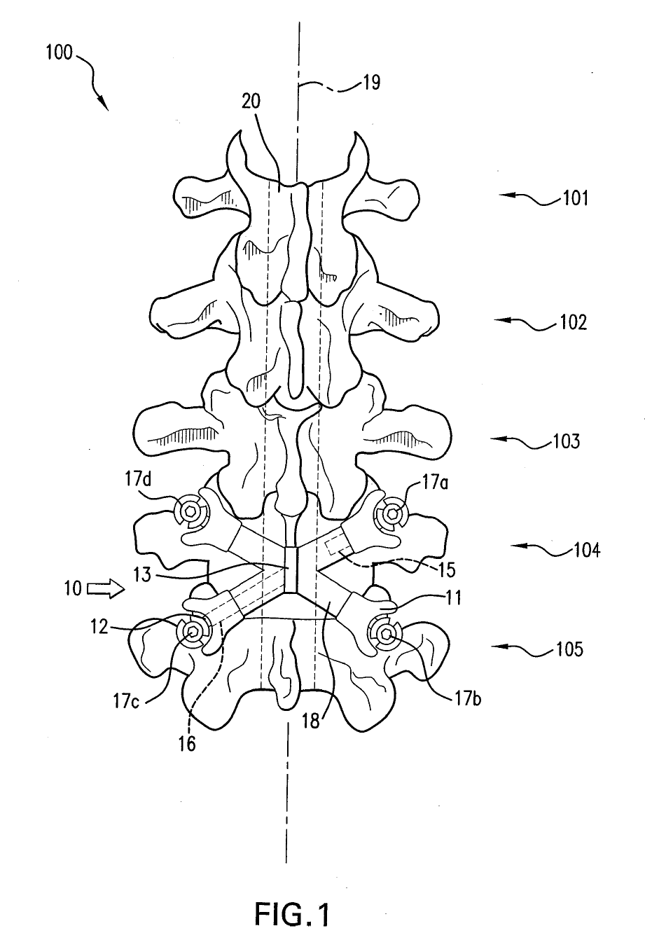

FIG. 1 illustrates an example of the present invention. There, a crossover connector 10 has been installed after removal of posterior portions of adjacent spinal vertebrae from the lumbar region of the spine. Visible in FIG. 1 is a portion of the spinal column 100 with five lumbar vertebrae numbered 101 through 105, four anchors numbered 17a to 17d, a crossover connector 10, and the spinal canal 20. The crossover connector 10 is shown with a main body 18, stretchable coupling 12, spacers 11, coupling passage 16, and central fin 13. The coupling passage 16 is present in each of the arms of the crossover connector although it is only illustrated in one of the arms. Likewise, the dowel 15 of the spacer 11 adjacent to anchor 17a is the only dowel illustrated even though a dowel may be present in each of the four illustrated spacers.

As shown in FIG. 1, pedicle screws or anchors 17 have been installed in the fourth 104 and fifth 105 lumbar vertebrae of the spinal column 100. Specifically, two anchors 17a and 17d have been placed in the pedicles of the fourth vertebrae and two anchors 17c and 17d have been placed in the pedicles of the fifth vertebrae. Now, to mimic functionality of the removed facet joints, a crossover connector 10 has been placed between the heads of the anchors 17. Extending from each of the four ends of the crossover connector 10 are end portions of the stretchable coupling 12. These end portions or anchor points of the stretchable coupling are coupled to the heads of the anchors 17 as shown in FIG. 1. Consequently, should the relative position of the fours anchors change and become spaced further apart, the stretchable coupling 12, which is positioned within the body 18 of the crossover connector 10, will stretch to some degree to accommodate this movement. More specifically, should anchor 17a move up and away from anchor 17b, perhaps due to the rotation or flexion of the spine, the stretchable coupling 12 extending from the main body 18 at anchor 17a and attached to anchor 17a will be stretched and will act to oppose the motion, pulling anchor 17a back towards anchors 17b and 17c. Likewise, when the other anchors move away from each other due to spinal column movement, the stretchable coupling may also act to oppose this movement and to pull the anchors back as well.

The crossover connector can also act on the anchors when the anchors are moved closer to one another, for example during extension of the spinal column. For example, when the lumbar vertebrae are moved such that anchor 17a is urged towards anchor 17b and anchor 17d is urged towards anchor 17c, the spacers 11 and main body 18 may compress to some degree and may act to retard movement of anchors 17a and 17d towards anchors 17c and 17d after the initial compression of the main body has occurred. This may have the effect of retarding further movement of the vertebrae anchors towards one another. When the anchors are not permitted to move any closer to one another, a minimum distance between the vertebrae in which the anchors are fixed, can be maintained.

When the anchors in the spinal column have moved relative to one another such that both compressive and tensile forces are being exerted on the crossover connector, portions of the main body of the crossover connector may be resisting compressive forces while portions of the stretchable coupling of the crossover connector may be resisting tensile or stretching forces. In so doing, the vertebrae to which the crossover connector are attached, may change position relative to one another and may also be supported to some degree by the crossover connector.

The spacers 11 of the crossover connector 10 may be removable or may be shaped and sized to accommodate the anchor heads and their relative positions. In other words, after the anchors are installed at their target sites, the distance between diagonally opposing anchor heads may be measured. The distance may then be used when selecting the proper sized spacer such that the crossover connector spans the space between opposing anchor heads. For example, if the space between anchors 17b and 17d is 4 cm and the main body is 2 cm, spacers 11 that are 8 mm in length may be chosen and coupled to the ends of the main body 18 such that the crossover connector fills 3.6 cm of the span between the anchors 17b and 17d. The additional 4 mm of space may remain to accommodate movement of the spine. Other dimensions may be used as well.

The central fin 13 of the crossover connector may be sutured or otherwise connected to paraspinal muscle in order to further hold the connector 10 in place. As such, fin 13 may define apertures for passage of suture. As can be seen, the central fin 13 is approximately aligned with the center line 19 of the spinal canal 20. However, this fin may be positioned in various locations depending upon the exact placement of the crossover connector and which vertebrae are connected. Moreover, it may not be present on the installed connector as it may have been removed by a practitioner prior to completion of the implantation procedure, perhaps because the practitioner finds that adequate space in the spinal area does not exist for the fin. As such, the central fin may be perforated to allow it to be removed at the discretion of the practitioner installing the crossover connector.

As can be seen, the stretchable coupling of the crossover connector may stretch or have a range of elasticity such that it may elastically deform during its anticipated ranges of motion. In other words, if the stretchable coupling is expected to stretch 2.0 cm from its installed position to the largest point in a range of motion, the material comprising the stretchable coupling may be elastically deformable from 0.0 to 3.0 cm. The material chosen may also become less stretchable outside of this elastic deformation region in order to better resist the forces placed on it during extreme movements. The material that comprises the coupling may also be chosen to resist millions of loading cycles that may occur once the connector is installed in the patient.

The main body 18 may be chosen from materials that resist compressive loads and that may not fatigue or fail when cyclical loading exceeds five million cycles. When compared, the coupling 12 may comprise a material with a higher elasticity than the main body 18 and the main body 18 may comprise a material that has a higher hardness than the coupling 12. These materials may be preferably resilient with good resistance to compressive loads and good resistance to fatigue from cyclical loading. Thus, it is preferable that the stretchable coupling be more compressible than the main body. In other words, when the same compressive pressure is exerted on the main body and the stretchable coupling, the stretchable coupling will compress more than the main body. Some suitable materials for stretchable couplings include sterile biocompatible materials such as, for example, nylon, polyethylene-terephthalate, silicone rubber, or suitable combinations thereof. Preferably, and as mentioned above, the materials that comprise the stretchable coupling are such that during anticipated ranges of motion, the stretchable coupling elastically deforms rather than plastically deforms. Some suitable materials for the main body include sterile biocompatible materials such as metallic materials and polymeric materials. Non-limiting examples of metallic materials include titanium, titanium alloys, chrome cobalt, stainless steel, or combinations thereof. Non-limiting examples of polymers include high-molecular weight polyethylene, polyether ketone, polycarbonate urethane, or combinations thereof. The central fin 13 may be made from the same material as the main body and it may be made from a different material as well.

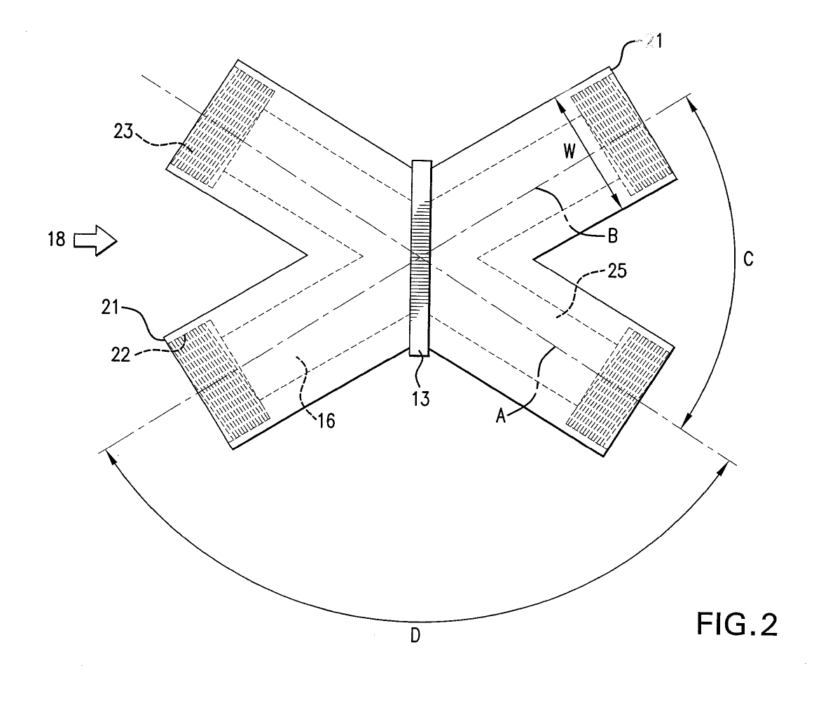

FIG. 2 illustrates an example of the enlarged main body 18 from FIG. 1. The main body 18 in FIG. 2 is illustrated as having four spacer receptacles 22, each with threads 23 and a central fin 13. A first coupling passage 16 and a second coupling passage 25 is also shown. Also labeled in FIG. 2 are center lines A and B, acute angle C and obtuse angle D. These center lines mark the centers of the coupling passages 16 and 25 while the acute and obtuse angles mark the angles defined by these center lines. As can be see, in the main body 18 of FIG. 2 the first passage and the second passage intersect one another. In other main bodies that embody the invention these passages may be partly or completely independent of one another. In other words, the passages may be connected to one another and may also be completely independent.

Screw threads 23 are shown in the ends of the main body 18. These threads may be sized and pitched to accommodate threads of the spacers that may be used to adjust the overall length of the main body 18. Other configurations other than threads may also be used to couple the spacers, which are not shown, to the main body 18. Once attached to the main body, the spacers may be rigidly held in place and may also be adjustable to some degree to accommodate movement.

The first and second coupling passages are shown within the main body. These passages are cylindrical but may be any suitable configuration. Within these passages a stretchable coupling, such as the stretchable coupling discussed above, may be placed. A single stretchable coupling having four ends may be placed in these passages. Two stretchable couplings, one coinciding with each center line (A,B), may also be placed therein. The center lines may be positioned relative to one another to form acute angle C and obtuse angle D. The main body 18 may be constructed such that these angles are fixed. The main body 18 may also be constructed such that angles C and D are changeable. If these angles are changeable, the main body may be adjusted to accommodate the actual position of anchors embedded in the vertebrae during a medical procedure. In other words, the main body may be contorted and flexed to accommodate the in-situ positions that it may need to take between embedded pedicle screws. In some embodiments, these adjustments may be fixed prior to implantation by mechanical locks and by changing the properties of the main body that comprises the crossover connector.

Portions of the crossover connector, including the main body, may contain a radio-opacifying agent within their structures to facilitate viewing the connector during and/or after the spinal device is implanted. Non-limiting examples of radio-opacifying agents are bismuth subcarbonate, bismuth oxychloride, bismuth trioxide, barium sulfate, tungsten, and mixtures thereof.

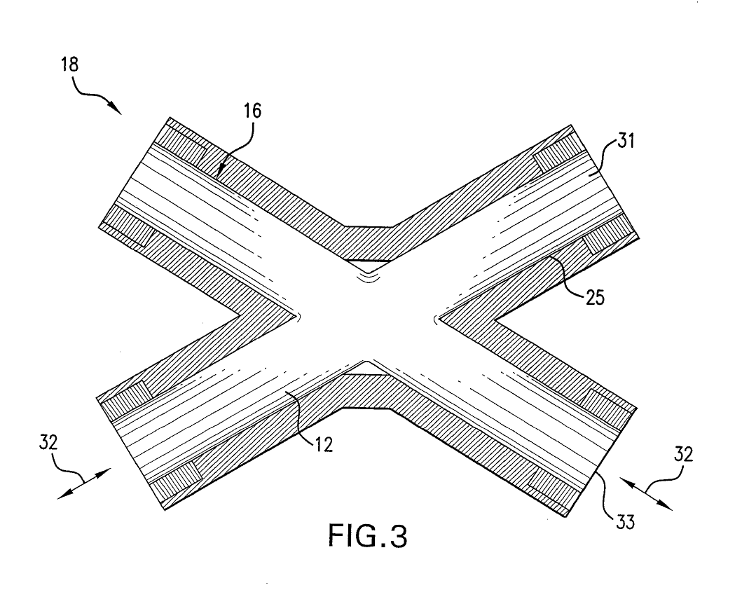

FIG. 3 is a sectional view of the main body 18 of FIG. 2. In FIG. 3, the first and second passages contain a stretchable coupling 12. As can be seen, the stretchable coupling 16 is a single unitary connector. The ends 33 of the stretchable coupling may extend from the main body as shown by arrows 32. They may be sized to extend from the main body when no load is placed on the stretchable coupling as well as when loads are placed on the connectors. Because of the unitary construction of the connector 12, when a load is placed on one of the anchor points of the connector by the anchor to which it is attached this load may be distributed to the other three anchor points. The stretchable coupling 12 may also be two or more chords, each positioned within the passages of the main body. These chords may each run the entire length of the passages and in some instances only a portion of the chords may run the entire length of the passages. In other words, if the chord consists of 100 strings only some of the strings may run the entire length of the passage. Moreover, while the stretchable body may be uniformly shaped across its entire length and cross-section, it may also be shaped differently to adjust its flexibility and strength. In other words, the shape of the stretchable coupling may be adjusted so that it provides less resistance to movement during the first few centimeters that it stretches and then provides more or significantly more resistance to movement when it stretches further than this.

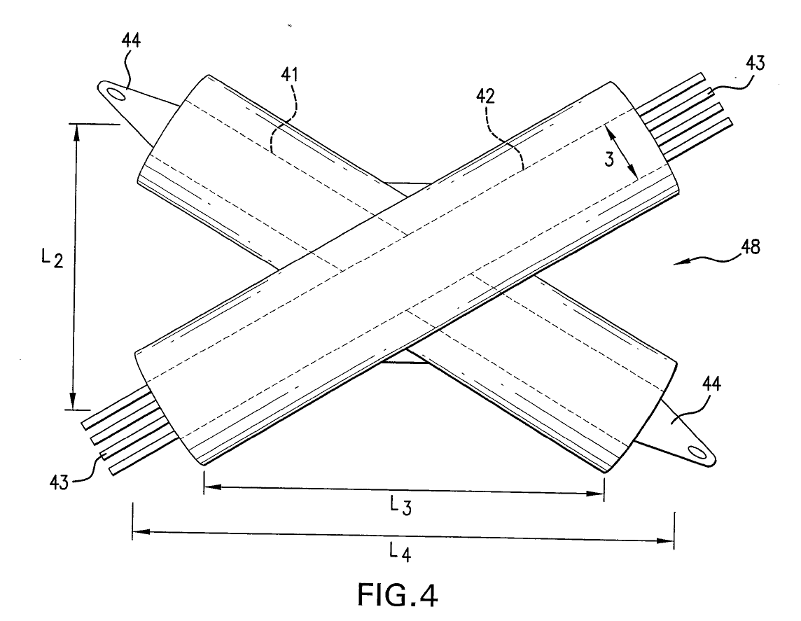

FIG. 4 shows the main body 18 of FIG. 1 when the first and second coupling passages (41 and 42) do not intersect but rather cross over each other. FIG. 4 also shows the ends of the stretchable couplings positioned within the passages 41 and 42. As can be seen, the ends 43 are shaped like individual chords, while the ends 44 are unitary and contain an orifice. The actual configuration of the ends or anchor points may depend upon the type of anchor to which they will be secured. Moreover, these anchor points need not be at the end of each of the chords. The illustrated anchor points 43 may be used if the ends need to be threaded on the anchor while the loop 44 may be used if the ends need to surround the anchor. Other configurations of the ends may also be suitable.

The outside length of the main body 18 along the acute angle side (L2) may be 2.5 cm while the inside length (L3) may be 3.5 cm and the outside length along the obtuse side (L4) may be 4.0 cm. Other dimensions and acute and obtuse angles may also be used. As such, the main body may be provided in various sizes and lengths and or the main body may be cutable or otherwise adjustable such that its overall size may be reduced prior to its use to accommodate the unique installation position in which it will be installed.

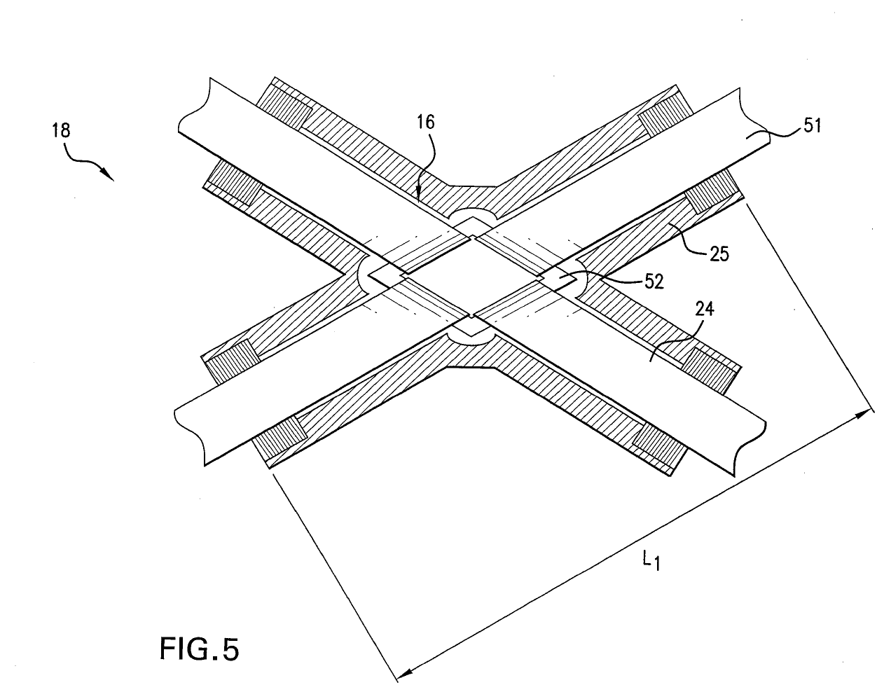

FIG. 5 shows the main body 18 having a stretchable coupling comprising four bands 51 and a frame 52. Each of the four bands 51 may be coupled to the frame 52 and may be positioned within and extend from the first and second passages of the main body. As to the ends coupled to the frame, this may accomplished by various methods including folding the end around the frame and mechanically securing the wrapped end back to the stretchable coupling in order to surround a portion of the frame. The frame may be comprised of any suitable sterile, biocompatible metallic or polymeric material. Even though a square frame is shown, other frame configurations may also be used.

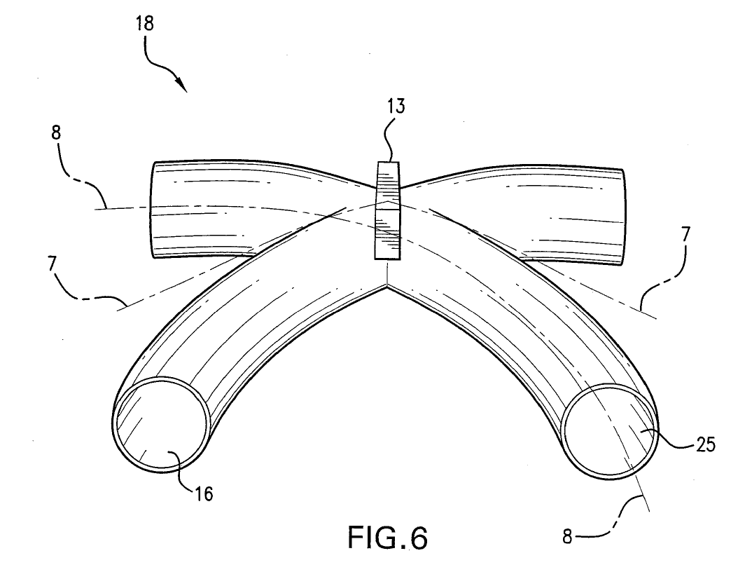

FIG. 6 shows a perspective view of the main body 18. As can be appreciated in this view, the main body 18 does not need to be planar and, in fact may be bowed in a preferred example in order to accommodate the spinal canal when it is installed. Also visible in FIG. 6 are the central fin 13 and the coupling passages 16 and 25.

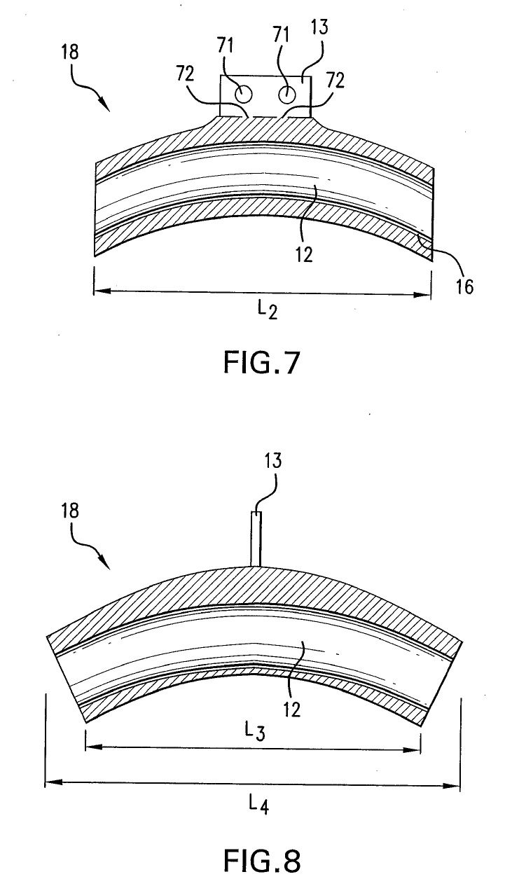

FIG. 7 is a sectional view taken along line 7-7 of FIG. 6. Visible in this figure are the suture holes 71 in the central fin as well as the stretchable coupling 12 and the passage 16. As can be seen in FIG. 7, the central passages need not be level but, rather, can be bowed in order to better accommodate the spinal canal. Also visible here are the serrations 72 along the bottom of the fin 13. These serrations 72 may be used as a fold line to bend the fin 13 or to remove it.

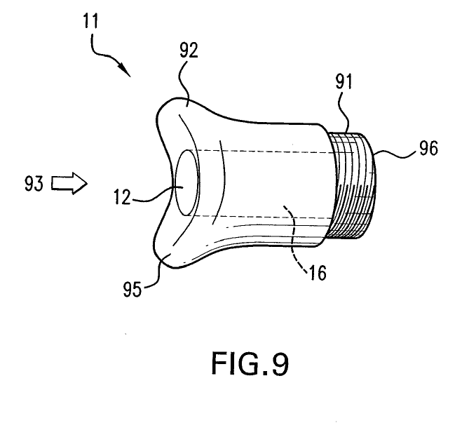

FIG. 8 is a sectional view taken along line 8-8 of FIG. 6. The fin 13 of main body 18 along with the stretchable coupling 12 are clearly visible.

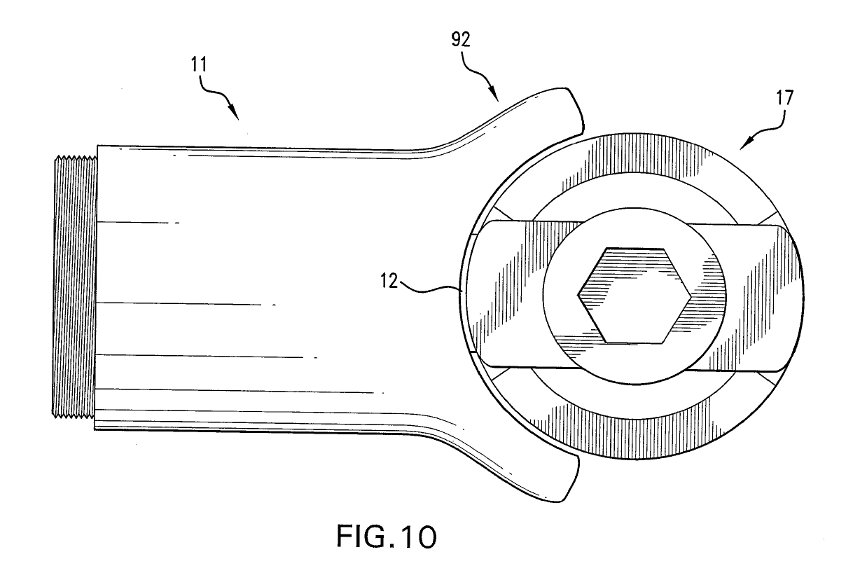

FIG. 9 is a prospective side view of a spacer as may be employed in accord with the current invention. As mentioned above, this spacer 11 and three others may be secured to the end of the main body 18 in order to adjust the length of the main body and also to accommodate the configuration of the anchors placed in the vertebrae. The spacer 11, which may form an open ended receptacle, may have anchor mating end or anchor abutment end 93 and dowel 91 with threads 96. These threads 96 may be threaded into the body 18 to secure the spacer 11 to the body 18. The coupling channel 16 and stretchable coupling 12 are illustrated in FIG. 9.

The anchor mating end 93 may be shaped and sized to seat against or otherwise meet an anchor. Here, the anchor mating end 93 includes a primary lip 92 and a second lip 95 that together form a semi-circle that can meet a correspondingly shaped anchor. These lips may be different sizes, as shown here, in order to accommodate anticipated larger loads being placed on the primary lip by the anchor. Once in place, the spacer may contact the anchor when compressive forces are being placed on the main body due to spinal column movement, perhaps during rotation and extension, and may not contact the anchor when tensile forces are being placed on the stretchable couplings from the anchor due to spinal movement, perhaps during flexion. These spacers may be provided to the practitioner as part of a kit such that the practitioner may choose the applicable sized spacer after the distance between anchors is known. The spacer 11 may be made from various sterile, biocompatible materials that are rigid and capable of being secured to the main body. These materials may also have different properties such that some are softer than others to provide initial cushioning and then firm resistance after the anchor and spacer engage one another during movement of the spinal column. In other words, the abutment face 93 may have an outer material with a first hardness and a second inner material with a second hardness wherein the second hardness is harder than the first hardness. Also, while threads are shown on the dowel, other attachment configurations may also be used.

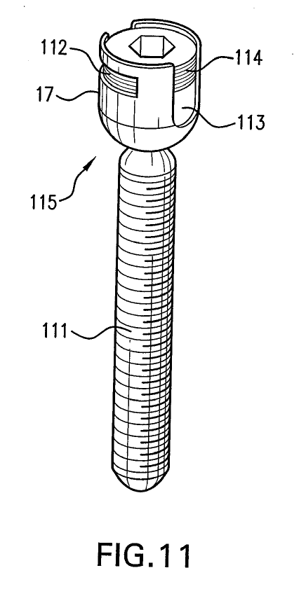

FIG. 10 is a plan view of a spacer 11 and anchor 17 in accord with the present invention. The anchor 17 in this figure is a pedicle screw with a hex screw head. As can be seen, the spacer 11 has uniform lips and is sized such that the mating end of the spacer does not surround the anchor head. The spacer 11 has threads at its body receiving end. The spacer may have various other abutment face configurations in order to accommodate the anchors being used and the layout and positioning of the pedicle screw installation.

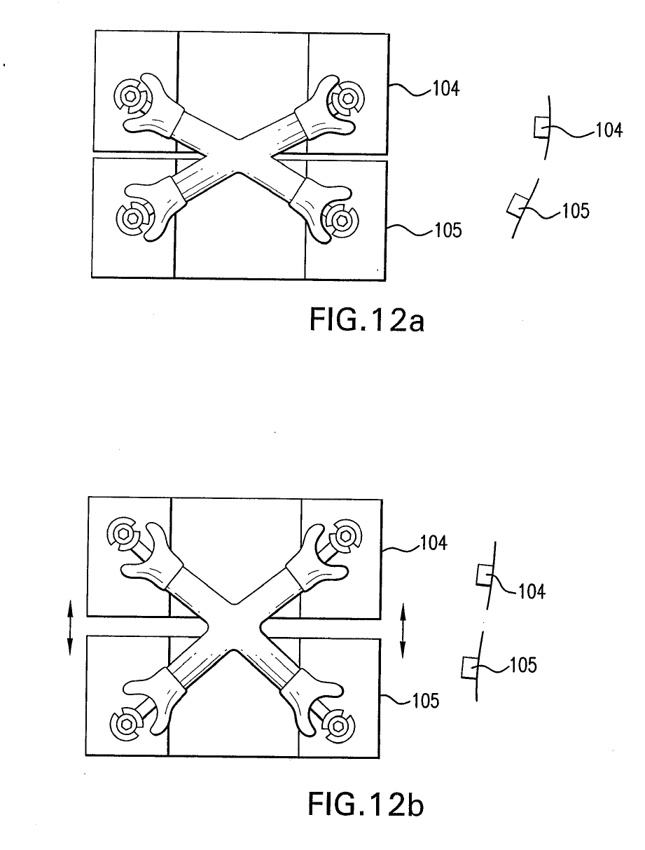

FIG. 11 is an isometric view of a pedicle screw that may be used as an anchor in accord with the current invention. The screw may have a bulb end 115 and a threaded end 111. The bulb end 115 may act as the attachment end for the anchor. An end of a stretchable coupling may be threaded through the opening 112 until it reaches the chamber 113 in the bulb 115. The hex head 114 may then be screwed down to secure the stretchable coupling within the void 113 and to the anchor. The pedicle screw may be made of titanium as well as from other metallic or polymeric materials. Other anchors, such as other types of screws and hooks may be used as well including, for example, connectors described in U.S. Pat. No. 6,626,908 and U.S. Pat. No. 5,474,555. Moreover, the anchors can be polyaxial to provide one or more degrees of motion between the anchors and the crossover connector.

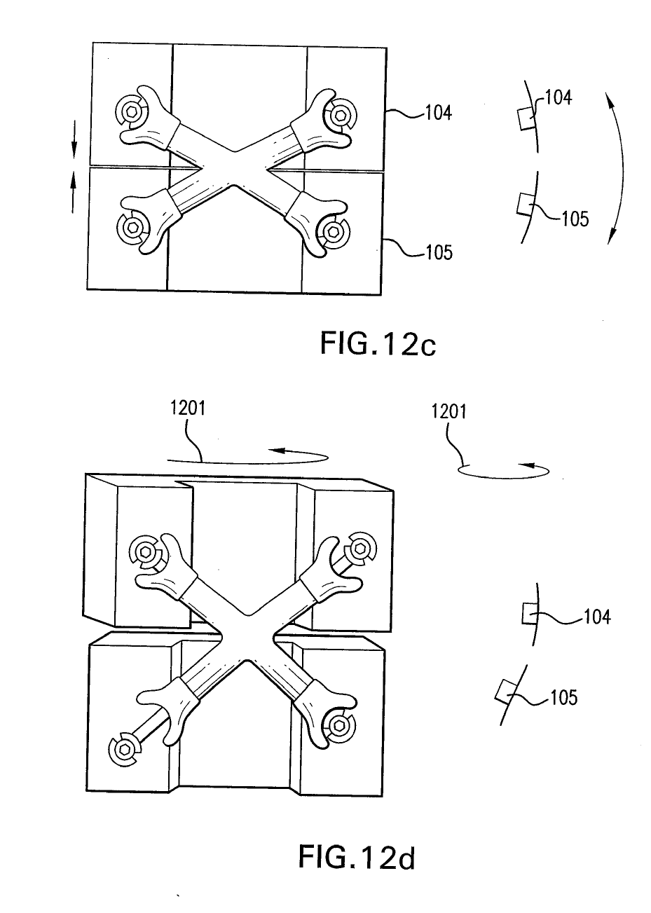

FIGS. 12a-12d are schematic block illustrations of a crossover connector in various spinal orientations. The illustrations on the left of each figure represents the third and fourth lumbar vertebrae coupled together with a crossover connector as seen from a posterior view. The right hand illustration represents a schematic orientation of the spine in which the connector is mounted as seen from a lateral view.

FIG. 12a reflects the anchors and connector in an installation orientation, which is the orientation of the spine during installation of the crossover connector. As can be seen from this figure, the stretchable coupling is coupled to the anchors and the spacers are not in contact with the anchors. This positioned may be obtained by placing the patient in a neutral position.

FIG. 12b shows the spacers moving apart from one another as the blocks representing the lumbar vertebrae are further apart in this figure. Here the vertebrae may be in a flexed orientation and the crossover connector may be resisting tensile forces acting to move the vertebrae of the spine apart from one another. As can be seen here, a tensile load is being placed on the stretchable couplings as these couplings are extending from and out of the ends of the spacers of the main body of the crossover connector.

FIG. 12c shows the anchors in a position where they have moved closer together and have a reached a point where the crossover connector prohibits further movement. By limiting further movement towards one another, the connector acts to maintain a certain distance between the adjacent vertebrae in which the anchors are embedded. The crossover connector is resisting compressive forces placed on it by the anchor heads. These compressive forces may be exerted on the spinal column when the spine is in extension as shown in the figure.

FIG. 12d shows some of the anchors moving away from the others. This may occur when the spinal column is rotated as shown by arrows 1201. As can be seen, the stretchable connector may be stretched along one diagonal of the connector and may not be as stretched along another diagonal. Moreover, one or more of the spacers may be in contact with the anchors while others are not. The exact orientation of the anchors will dictate how the connector will be acted upon and will be resisting movement. Thus, the crossover connector may be resisting tensile and compressive forces at the same time.

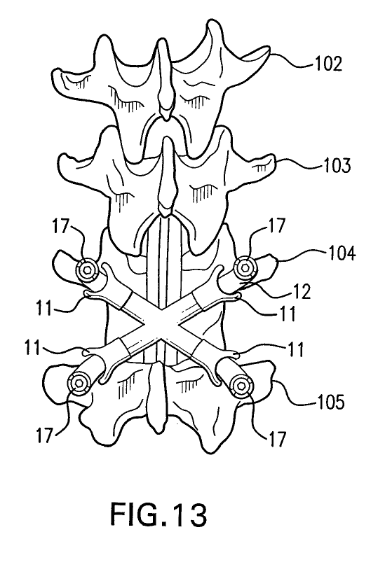

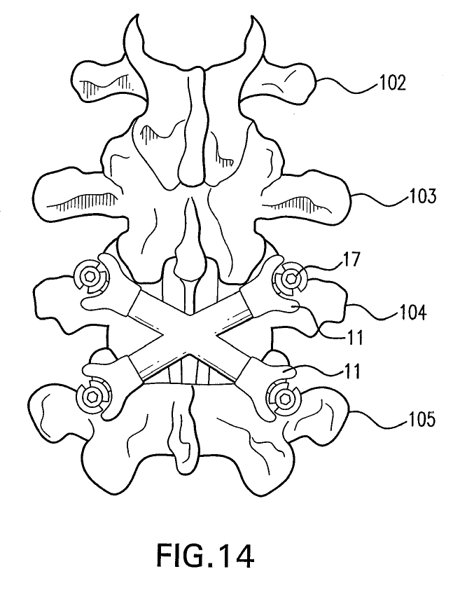

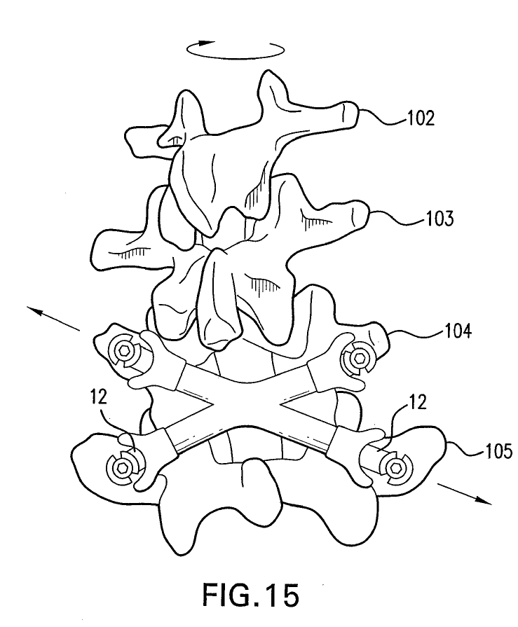

FIG. 13 is posterior view of a spinal column and crossover connector corresponding to FIG. 12b when the spine is being flexed. FIG. 14 is a posterior view of the spinal column and crossover connector corresponding to FIG. 12c when the spine is being extended. FIG. 15 is a posterior view of the spinal column when the spine is being rotated.

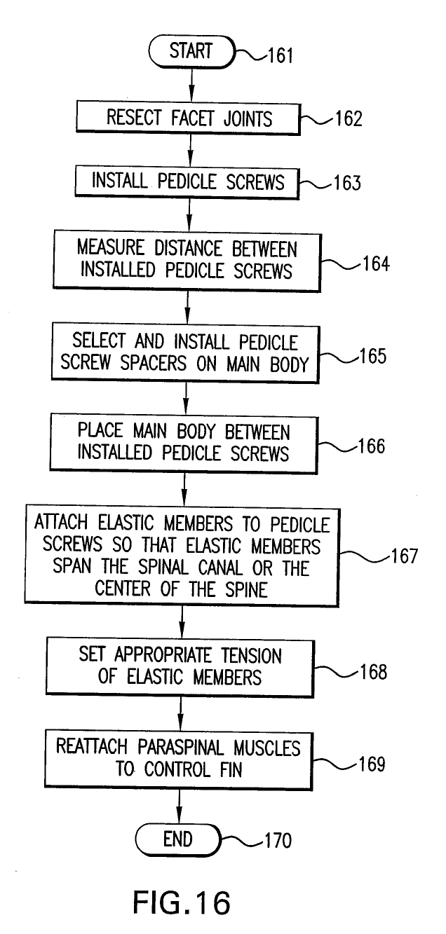

FIG. 16 is a chart of action that may be performed in accord with the present invention. These actions may include resecting facet joints of the vertebrae as shown at 162 and installing pedicle screws as shown at 163. Once the pedicle screws are installed the distance between the screws may be measured and the appropriate spacers may be chosen for attachment to the main body of the connector based upon the measurements as indicated at 164 and 165. When the spacer is selected the practitioner may be offered various lengths of spacers and spacers with various attachments heads in the kit along with written instructions in order to select and accommodate the orientation and type of anchors utilized. The spacers may be coupled to the main body and the main body and spacers may be positioned between pedicle screws as shown at 166. The elastic members resident within the main body may then be coupled to pedicle screws as shown at 167. The appropriate tension of the elastic members may then be set as shown at 168. The central fin may also be attached to the surrounding area or it may be removed. This is shown at 169.

When carrying out these steps it is preferred that the patient is placed in a prone position and the facet joints at the affected area are carefully identified and exposed. As part of a wide laminectomy procedure, the facet joints may be completely resected such that the resection can be as wide as pedicle to pedicle. Moreover, when pedicle screws are used as anchors, they may be placed in the respective vertebrae without compromise of the super adjacent facet joint complexes. When implanted, the crossover connector may span two or more vertebrae in the spine and may be located outside the intervertebral disc spaces in the spinal column. The main body generally encases the stretchable coupling to protect the neural elements of the spine from contacting the main body. The paraspinal muscles may be attached to the main body by suturing the paraspinal muscles to the fin of the body.

The present invention may be used for facet joint-related conditions. For example, it may be used for conditions where the patient complains of leg pain and/or back pain and where the pain has been identified as being caused by the lumbar facet joints themselves, or by neural compression resulting from hypertrophic, arthritic facet joints. Non-limiting examples of such conditions include spinal stenosis, lumbar lateral recess stenosis, neuroforaminal stenosis, lumbar facet joint syndrome, lumbar facet cyst formation. The present invention may also be used for discogenic pain syndromes when accompanied by posterior element disease. Further, the invention may be used in conjunction with disc arthroplasty, such as constrained anterior disc replacement, since the spinal devices replace the posterior elements of the spine. This may be advantageous since moderate or advanced facet disease, previous facetectomy, or other prior destabilizing procedures may be a contraindication for prosthetic discs. As such, the present invention may be used in conjunction with a disc prosthesis or a disc nucleus replacement disposed between adjacent vertebra in a spinal column.

The foregoing description and examples have been set forth merely to illustrate the invention and are not intended as being limiting. Each of the disclosed aspects and embodiments may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. A system for linking vertebrae anchors in vertebrae of a spinal column having a spinal canal, the system comprising:
a connector having a main body and an elastically stretchable coupling,
the connector and the elastically stretchable coupling being sterile,
the elastically stretchable coupling elastically stretchable from a first length to a second length,
the elastically stretchable coupling having a first anchoring point and a second anchoring point,
the main body being bowed, having a length, a first end and a second end,
the first end configured to abut a first vertebrae anchor head,
the second end configured to abut a second vertebrae anchor head,
the length sufficient to span the spinal canal when the main body is positioned between the first vertebrae anchor head and the second vertebrae head and the first anchor head is positioned on a first vertebrae and the second anchor head is positioned on a second vertebrae.

2. The system of claim 1, wherein the connector contains a passage and wherein the elastic coupling is positioned within the passage.

3. The system of claim 1, wherein the main body defines an acute angle and an obtuse angle.

4. The system of claim 1 wherein the stretchable coupling further comprises a third anchoring point and a fourth anchoring point.

5. The system of claim 1 wherein the stretchable coupling further comprises a frame.

6. The system of claim 1 wherein a spacer is coupled to the first end of the main body.

7. The system of claim 6 wherein the spacer has an open end receptacle configured to abut an anchor head.

8. The system of claim 6 wherein the spacer comprises two materials, the first material having a greater hardness than the second material.

9. The system of claim 1 wherein the main body is x-shaped and wherein the main body defines a first passage and a second passage.

10. The system of claim 1 wherein the main body has an external surface and wherein the external surface has a fin disposed thereon.

11. The system of claim 1 wherein the anchors are pedicle screws.

12. The system of claim 11 wherein the anchoring points of the stretchable coupling are threaded through the pedicle screws.

13. A method of linking anchors on spinal vertebrae of a spinal column, the method comprising:
installing a first anchor into a spinal vertebrae;
installing a second anchor into a spinal vertebrae;
positioning a connector having a main body and a stretchable coupling between the first anchor and the second anchor after the anchors have been installed; and
securing a stretchable coupling to the first anchor and the second anchor such that the stretchable coupling crosses over a longitudinal center axis of the spinal column containing the vertebrae,
wherein after the stretchable coupling is secured to the first anchor and the second anchor, the main body of the connector does not contact at least one of the installed anchors.

14. The method of claim 13 further comprising measuring the distance between the installed first and second anchor and selecting and installing a spacer on the main body.

15. The method of claim 13 further comprising:
suturing paraspinal muscles to a fin of the main body.

16. A spinal crossover connector comprising:
a sterile body comprising a biocompatible material,
the body defining a first passage with a first end and a second end, the first end having an abutment face configured to abut a spinal anchor, the second end having an abutment face configured to abut a second spinal anchor;
a stretchable coupling in the first passage, the body having a lower compressibility than the first stretchable coupling, the stretchable coupling extending from within the first passage to outside of the first end; and
a second passage, the second passage being non-parallel to the first passage, the second passage containing a stretchable coupling.

17. The crossover connector of claim 16 wherein the body defines the first passage and the second passage.

18. The crossover connector of claim 16 further comprising a spacer, the spacer coupled to an end of the first passage.

19. The crossover connector of claim 17 wherein the spacer defines a lumen.

20. The crossover connector of claim 16 wherein the body is reconfigurable to define a first acute angle in a first position and a second acute angle in a second position, the second acute angle being different from the first acute angle.

21. The crossover connector of claim 16 wherein the stretchable coupling in the first passage is coupled to the stretchable coupling in the second passage.

22. The crossover connector of claim 16 wherein the body is bowed.

23. The crossover connector of claim 16 wherein the stretchable coupling comprises a frame.

24. The crossover connector of claim 16 wherein the stretchable coupling comprises four anchoring points.
